(19) United States
(12) Patent Application Publication
(10) Pub. No.: US 2007/0148268 A1
(43) Pub. Date: Jun. 28, 2007

(54) CHEMICAL COMPOUNDS AND THEIR USE AS THERAPEUTIC AGENT AND MEANS OF QUALITY ASSESSMENT OF HERBAL MEDICINES

(75) Inventors: Chun Feng QIAO, Hong Kong (CN); Quan-Bin HAN, Hong Kong (CN); Jing-Zheng SONG, Hong Kong (CN); Shi-Fu MO, Hong Kong (CN); Ling-Dong KONG, Nanjing (CN); Hsiang-Fu KUNG, Hong Kong (CN); Hong-Xi XU, Hong Kong (CN)

Correspondence Address: EVAN LAW GROUP LLC, 600 WEST JACKSON BLVD., SUITE 625, CHICAGO, IL, 60661, US

(73) Assignee: Hong Kong Jockey Club Institute of Chinese Medicine Limted, Hong Kong (CN)

(21) Appl. No.: 11/614,090

(22) Filed: Dec. 21, 2006

Related U.S. Application Data
(60) Provisional application No. 60/752,960, filed on Dec. 23, 2005.

(51) Int. Cl. A61K 36/23; A61K 31/7048

(52) U.S. Cl. 424/777; 514/27; 536/17.1

(57) ABSTRACT

Two novel benzofuran glycosides, named psoralenoide and isopsoralenoide, isolated and purified from the seeds of Psoralea corylifolia. The compounds are useful as therapeutic agents for diseases defined under TCM and as a more accurately means of quality control and assessment for Psoralea corylifolia, an important herbs used in TCM.

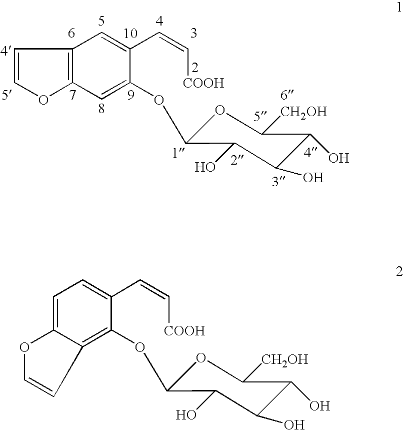

### CROSS REFERENCE TO RELATED APPLICATIONS

This application claims priority to U.S. Provisional Application No. 60/752,960, filed Dec. 23, 2005, the contents of which are hereby incorporated by reference.

### FIELD OF THE INVENTION

This invention relates to new chemical compounds isolated and purified from herbal medicines. Particularly, it relates to two previously unknown compounds which are glycosides of psoralen and isopsoralen, respectively, and relates to a method of using the two compounds as therapeutics agents and as a means for quality control and evaluation of the herbal medicine Fructus Psoraleae.

### BACKGROUND OF THE INVENTION

Fructus Psoraleae, the dried fruits of Psoralea corylifolia L., is one of the most popular traditional Chinese medicines and is officially listed in the Chinese Pharmacopoeia. This herbal medicine has been used for the treatment of “enuresis, pollakiuria, weak kidney, and pain and cold in the waist and knees,” a group of diseases defined under the Traditional Chinese Medicine (TCM). It has always attracted research interest and, in the year of 2005 alone, over ten articles was published, all focusing on its quality analyses[1-3], photochemistry[4-8], and bioactivities[9-16].

Previous photochemistry revealed that Fructus Psoraleae contains psoralen, isopsoralen, psoralidin, corylifolin, isobavachalcone, corylin, and backuchiol. Among them, two major coumarins, psoralen and isopsoralen, are commonly used as quality markers, or in other words, the coumarins are regarded as the main active ingredients of the herbal medicine. All previously reported quality control methods for this important herbal medicine were based on the coumarins. It was unknown prior to the present invention that any benzofuran glycosides would exist in the herb Fructus Psoraleae.

### SUMMARY OF THE INVENTION

As an object of the present invention, there is provided two novel chemical compounds and their functional derivatives. The two parent compounds were previously unknown and were newly isolated and purified from the herbal medicine Fructus Psoraleae. They are benzofuran glycosides of formula (1) and (2), respectively. Based on the teaching of the present invention, people of ordinarily skill in the art would understand that the benzofuran glycosides are one of the main active ingredients contributing to the herbal medicine's therapeutic effects. The present invention also provides a method for isolating and purifying the benzofuran glycosides from herbals.

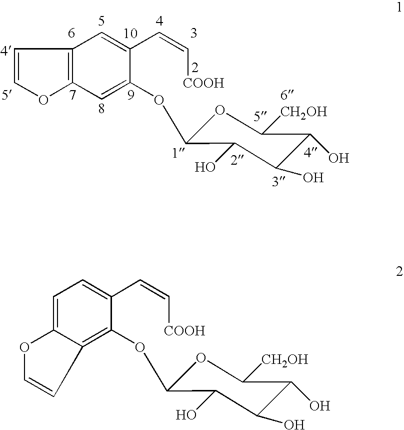

As another object of the invention, there is provided a method for quality control and evaluation of the herbal medicine Fructus Psoraleae using the two compounds as chemical markers, alone or combined with other previous known chemical markers. This quality evaluation method yields significantly better repeatability and consistency than any previously known methods.

### BRIEF DESCRIPTION OF DRAWINGS

FIG. 1 shows the HPLC-ESI-MS spectra of compounds 1 and 2.

FIG. 2 shows key HMBC correlations of compounds 1 and 2.

FIG. 3 depicts chemcial transformation of compounds 1 and 2 to compounds 3 and 4.

FIG. 4 is typical HPLC chromatogram of compounds 1-4 in Fructus Psoraleae (A: reference standards, B: sample No. 23, C: sample No. 1)

FIG. 5 is the contents of psoralen, isopsoralen, psoralenoside, and isopaosrelnoside for the 23 samples examined in the present invention.

FIG. 6 list the total contents of psoralen and isopsoralen calculated for the 23 samples according to the present invention.

FIG. 7 shows a possible route by which compounds 1 and 2 are converted to compounds 3 and 4.

### DETAILED DESCRIPTION OF THE INVENTION

Isolation of the New Compounds

Equipment Setup: ESI-MS was recorded on a VG Auto Spec-3000 spectrometer. ID-and 2D-NMR spectra were run on a Brucker AM-400 and a DRX-500 instrument with TMS as internal standard, respectively. The HPLC-ESI-MS analyses were carried out using an Agilent 1100 series which chromatographic system consisted of a binary pump, photodiode array detector (DAD), coupled to an Agilent MSD-Trap SL through a Brucker atmospheric pressure chemical ionization (APCI) interface. The APCI-MS spectra were acquired over a range of m/z 100-900 in positive ion mode. The temperature of dry gas and APCI were set at 350 □and 400 □, respectively. The nebulizer gas pressure was 50 psi and the dry gas flow rate was 51/min. The HPLC system was directly connected to the MS without stream splitting. The injection volume of sample was 10 μl. The Agilent 1100 series HPLC system equipped with Zorbax® XDB-C₈ analytical column (4.6×150 mm, 5 μm, Agilent Technologies, U.S.A.), a C₁₈ guard column (4.6×12.5 mm, 5 μm, Agilent Technologies, U.S.A.), a DAD, and an Alltech ELSD 2000 detector (USA) was setup for the analysis. The analysis was performed at 20 ° C. during the whole process. The mobile phase was a mixture of methanol and 0.1% acetic acid at a flow rate of 1.0 ml/min. Linear gradient elution from 10% to 88% methanol (v/v) in 40 min was applied. Herbal Material: The dried fruits of P. corylifolia were purchased in Shenzhen, China, and authenticated by Dr. C. F. Qiao, Chinese Medicine Laboratory, Hong Kong Jockey Club Institute of Chinese Medicine, Hong Kong, China. A specimen (No CMED-0178-26) was deposited at the Chinese Medicine Laboratory, HKJCICM.

Extraction and Isolation: The herbal material (1 kg) was processed into powder, which was then refluxed twice with 5 L EtOAc. The residue was then refluxed twice each with 5 L MeOH. The MeOH extracts were combined. The combined extract was filtered and evaporated to dryness by rotary evaporation below 60° C. under reduced pressure. The dried residue was suspended in water. The water solution was loaded on a Diaion HP-20 macroporous resin column, eluting with water. The glycoside fraction was collected based on the absorption peak as detected by a built-in detector of the HPLC equipment. Separation of the target fraction on an ODS column eluting with 5% MeOH was repeated until yielding two compounds, which were subsequently identified as benzofuran glycosides, whose structure are defined by formula (I) and (II), respectively. These two compounds were further purified on a Sephadex LH-20 column eluted with MeOH and subject to MS and NMR analyses for structural identification.

Identification of the New Compounds

By using the foregoing disclosed method, the two new compounds were obtained and shown as major peaks in the HPLC chromatogram (never be reported before). Interestingly, when the herbal material was extracted with boiling water, these two compounds were not present in the extract, even if they have a high polarity close to sugar.

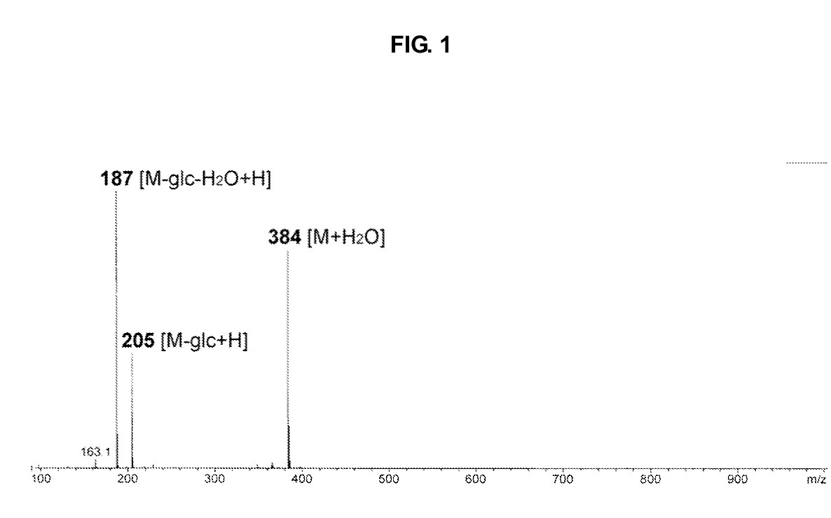

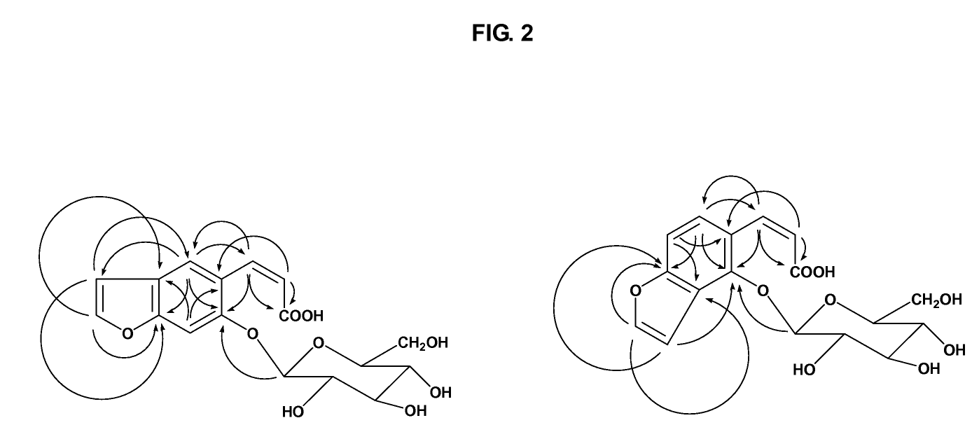

The analyses with high performance liquid chromatography electrospray ionization mass spectroscopy (HPLC-ESI-MS) showed that the two compounds had the same MS spectra, in which three major ion peaks were displayed clearly. As shown in FIG. 1, the three peaks were at m/z 384, m/z 205, and m/z 187, corresponding to [M+H₂O]⁺, [M-glc+H]⁺, and [M-glc-H₂0+H]⁺(i.e., [psoralen/isopsoralen+H]⁺), respectively. In the high resolution electrospray ionization mass spectroscopy (HR-ESI-MS), compound 1 showed the [M+K]⁺ peak at m/z 405.0601, in accord with the molecular formula C₁₇H₁₈O₉ (calculated. 405.0582 for C₁₇H₁₈O₉K), and compound 2 displayed the [M+Na]⁺ peak at m/z 389.0869, indicating the same molecular formula C₁₇H₁₈O₉ (calculated. 389.0843 for C₁₇H₁₈O₉Na). These results suggested that compounds 1 and 2 are glycosides of psoralen and isopsoralen, respectively. This conclusion was confirmed by the detailed analyses of their NMR spectra. The ¹H and ¹³C NMR spectra of compound 1 exhibited two groups of signals in consistent with the structural moieties of psoralen and glucose [19]. The glucose was located at C-9 by the key heteronuclear multiple bond connectivity (HMBC) correlation (FIG. 2) between the anomeric proton H″-1 (δ4.98, d, J=7.6 Hz) with C-9 (δ155.1). Furthermore, the glucose was β-orientated according to the coupling constant J=7.6 Hz. A similar benzofuran glucoside, namely psoralic acid O-β-D-glucopyranoside, had been reported from the seeds of P. plicata [19], differing from compound 1 only at the trans-cinnamic acid residue. The coupling constant (J) of the trans-coupled olefinic protons was 16.0 Hz, while that of compound 1 was 12.4 Hz, which suggested that the cinnamic acid moiety was cis-coupled. This deduction was confirmed by the clear NOE effect arising from these two cis-coupled protons (δ6.07 and 6.95, d, J=12.4 Hz) in the ROESY spectrum of compound 1. Therefore, compound 1 was determined to be the cis-isomer of psoralic acid O-β-D-glucopyranoside, named psoralenoside. In the same way, compound 2 was determined to be cis-isomer of isopsoralic acid O-β-D-glucopyranoside, and named isopsoralenoside. Their NMR data were unambiguously assigned on the basis of extensive 2D NMR analysis (Table 1).

TABLE 1
¹H and ¹³C NMR data of 1 and 2 in CDCl₃
(δ in ppm, J in Hz)

| | |
|---|---|
| 1 | 2 |

| C | ¹H | ¹³C | ¹H | ¹³C |
|---|---|---|---|---|
| 2 | | 177.1 | | 178.0 |
| 3 | 6.07, d, 12.4 | 127.8 | 6.07, d, 12.4 | 129.0 |
| 4 | 6.95, d, 12.4 | 129.8 | 7.03, d, 12.4 | 128.7 |
| 5 | 7.88, s | 123.1 | 7.67, d, 9.2 | 128.0 |
| 6 | | 123.8 | 7.20, d, 9.2 | 108.9 |
| 7 | | 157.2 | | 158.6 |
| 8 | 7.36, s | 100.6 | | 122.5 |
| 9 | | 155.1 | | 150.6 |
| 10 | | 124.9 | | 125.8 |
| 4′ | 6.75, d, 2.0 | 108.1 | 7.18, d, 2.0 | 107.5 |
| 5′ | 7.65, d, 2.0 | 146.9 | 7.65, d, 2.0 | 146.7 |
| 1″ | 4.98, d, 7.6 | 103.7 | 4.95, d, 7.6 | 106.6 |
| 2″ | 3.53-3.55, m | 75.5 | 3.54-3.56, m | 76.5 |
| 3″ | 3.51-3.53, m | 78.8 | 3.45-3.49, m | 79.2 |
| 4″ | 3.47-3.49, m | 72.0 | 3.45-3.49, m | 72.4 |
| 5″ | 3.49-3.51, m | 78.7 | 3.45-3.49, m | 79.1 |
| 6″ | 3.92, dd, 12.0, | 63.1 | 3.83, dd, 12.0, | 63.6 |
| | 3.0; 3.73, dd, | | 3.0; 3.72, dd, | |
| | 12.0, 5.6 | | 12.0, 5.6 | |

Nine other compounds were also isolated from the herbal materials, which were known. By comparing with the reported data [3,5], they were identified to be psoralen, isopsoralen, psoralidin, corylifolin, corylin, corylifolinin, isobavachalcone, corylifol A, and bakuchiol.

Obtaining benzofuran glycosides from herbs proved to be challenging work. In a previously reported phytochemical study on the seeds of Psoralea plicata [19], several similar benzofuran glycosides were reported. These glycosides, however, were obtained as acetates. Acetylization had to be carried out to improve the separation by changing the chemical and chromatographic properties of these glycosides. In other words, those glycosides obtained did not have free hydroxyl groups. In the present invention, the glycosides were first isolated by repeated column chromatography (CC) on ODS. Although the HPLC analyses (equipped with UV detector) showed only one peak, they were proved not pure by NMR analysis. The impurity was revealed by evaporative light scattering detector (ELSD), and was finally removed by CC on Sephadex LH-20 eluting with MeOH.

Therefore, the present invention has not only provided two new compounds but removed the uncertainty regarding the nature of benzofuran glycosides reported previously. Based on the biosynthesis relationship between these glycosides and their aglycone derivatives (psoralen and isopsoralen), it can now reasonably conclude that the cinnamic acid group is cis-form. The trans-form of these reported glycosides might be derived from the related acetylization. This conclusion is entirely consistent with the discovery of the cis-isomeric glycosides (compounds 1 and 2) of the present invention.

Effects of Extracting Methods on the Yielding of Glycosides:

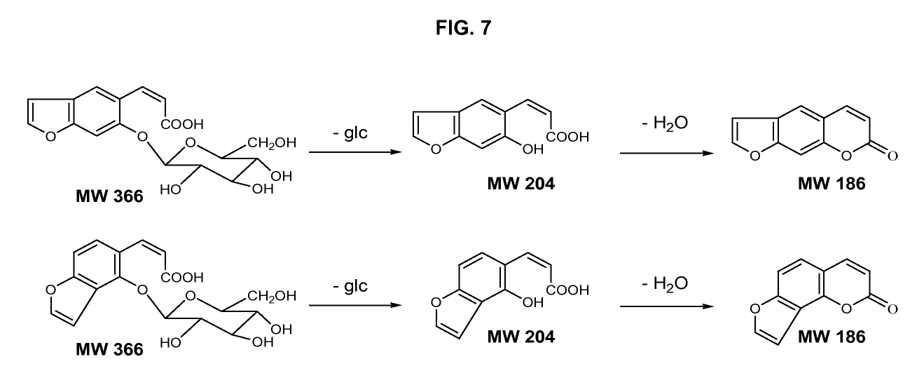

According to the present invention, the yielding of glycoside compounds 1 and 2 varies according to the particular method employed for extraction. Specifically, if the herbal material was extracted using water or a water/alcohol mixture as the solvent, compounds 1 and 2 were converted to compounds 3 and 4 to varying degrees, respectively. While not intended to be bound to the theory, applicants believe that this conversion may take place according to the scheme shown in FIG. 7. However, this conversion would not occur if, prior to the water extraction, the herbal material (in the powder form) was pretreated (i.e., moistened and then dried up) with an organic solvent, for example, hexane, cyclohexane, Petroleum ether, benzene, chloroform, Diethyl ether, ethyl acetate, n- butanol, acetone, ethanol, acetonitrile, etc. By preventing the conversion, this pretreatment led to the maximum yield for compounds 1 and 2. Accordingly, when compounds 1 and 2 are the desired active ingredients for treating a particular disease defined under the TCM, one may pretreat the herbal material with an organic solvent before the water or water/ethanol extraction. Similarly, this pretreatment should be employed for isolation and purification of compounds 1 and 2 in order to further process them into a pharmaceutical composition for the usages that rely on compound 1 and/or compound 2.

Use of the New Compounds as Chemical Markers for Quality Assessment

Reagents and Materials: Twenty-three batches of raw herbal material of Fructus Psoraleae were collected from different regions of China and they were authenticated as genuine P. corylifolia according to their morphological characteristics. The reference chemical standards of psoralen (batch No. 110739-200309) and isopsoralen (batch No. 110738-200410) were purchased from the National Institute for the Control of Pharmaceutical and Biological Products of China (Beijing, China). Psoralenoside and isopsoralenoside were isolated and purified as described in the foregoing. The purities of the chemical references were determined to be higher than 98% by HPLC analysis. HPLC grade methanol and acetic acid were purchased from International Laboratory (Nevada, U.S.A.). HPLC grade water was prepared by Millipore Milli-Q SP water purification system.

Equipment Setup: The Agilent 1100 series HPLC system equipped with a Zorbax® XDB-C₈ analytical column (4.6×150 mm, 5 μm, Agilent Technologies, U.S.A.), a C₁₈ guard column (4.6×12.5 mm, 5 μm, Agilent Technologies, U.S.A.) and a photodiode array detector (DAD) was set up for the analysis. The analysis was performed at 20 ° C. during the whole process. The mobile phase was a mixture of methanol and water (containing 0.1% acetic acid) at a flow rate of 1.0 ml/min. Linear gradient elution from 25% to 60% methanol (v/v) in 30 min was applied. The detection wavelength was set at 246 nm.

Standard solutions: The stock solutions were prepared in methanol at the concentrations of 0.636 mg/ml (psoralen), 0.588 mg/ml (isopsoralen), 0.611 mg/ml (psoralenoside) and 0.587 mg/ml (isopsoralenoside). The stock solutions were diluted and mixed in 5 ml volumetric flasks to yield a series of standard solutions with different concentrations for the validation of linearity.

Sample Solutions: Samples were pulverized to power, which was screened through 355 μm sieves. The fine powder (1 g) was accurately weighed, and mixed with 50 ml methanol in a flask (100 ml). The flask was weighed again. Then, the powder was refluxed in methanol for 4 hours. After cooling, methanol was added to make up to the initial weight. The supernatant fluid was filtered through a syringe filter (0.45 μm). 5 μl of this solution was injected into the HPLC system for analysis.

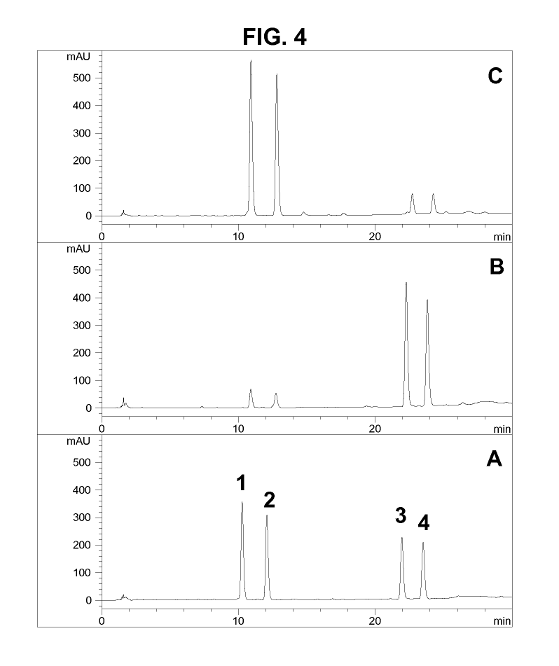

The Experimental Data as Basis for Herbal Quality Assessment: Table 2 lists the 23 samples of Fructus Psoraleae collected from different regions of China that used in the example. For each sample, the HPLC chromatogram was generated at wavelengths of 246 nm (shown in FIG. 4). The peaks corresponding to psoralenoside (compound 1), isopsoralenoside (compound 2), psoralen (compound 3), and isopsoralen (compound 4) in the HPLC chromatogram were identified by comparing with the reference chemical standards.

TABLE 2
Contents (%, w/w) of compounds 1-4 in 23 Samples of Fructus Psoraleae

| Sample | Purchased markets | | | | |
|---|---|---|---|---|---|
| No. | (growth area) | 1 | 2 | 3 | 4 |
| 1 | Chongqing (Chongqing) | 3.02 | 2.68 | 0.05 | 0.05 |
| 2 | Chongqing (Chongqing) | 2.55 | 2.39 | 0.15 | 0.16 |
| 3 | Nanjing, Jiangsu (Yunnan) | 2.33 | 1.99 | 0.18 | 0.16 |
| 4 | Chongqing (Chongqing) | 2.24 | 1.96 | 0.14 | 0.10 |
| 5 | Shenzhen, Guangdong (Sichuan) | 2.07 | 1.76 | 0.25 | 0.22 |
| 6 | Hong Kong (unknown) | 1.95 | 1.66 | 0.30 | 0.27 |
| 7 | Nanjing, Jiangsu (Yunnan) | 1.89 | 1.56 | 0.29 | 0.27 |
| 8 | Nanjing, Jiangsu (Anhui) | 1.87 | 1.59 | 0.26 | 0.23 |
| 9 | Hong Kong (unknown) | 1.84 | 1.51 | 0.25 | 0.22 |
| 10 | Hong Kong (Guizhou) | 1.75 | 1.46 | 0.35 | 0.34 |
| 11 | Shuozhou, Shanxi (Liaoning) | 1.75 | 1.49 | 0.41 | 0.36 |
| 12 | Nanjing, Jiangsu (Guangxi) | 1.73 | 1.45 | 0.37 | 0.32 |
| 13 | Xinxiang, Henan (Henan) | 1.69 | 1.44 | 0.42 | 0.38 |
| 14 | Hong Kong (Guangxi) | 1.55 | 1.32 | 0.49 | 0.45 |
| 15 | Shuozhou, Shanxi (Liaoning) | 1.55 | 1.39 | 0.46 | 0.43 |
| 16 | Shenzhen, Guangdong (unknown) | 1.32 | 1.10 | 0.44 | 0.40 |
| 17 | Wuhan, Hubei (unknown) | 1.29 | 1.03 | 0.48 | 0.40 |
| 18 | Shenzhen, Guangdong (unknown) | 1.02 | 0.85 | 0.55 | 0.55 |
| 19 | Hong Kong (unknown) | 0.95 | 0.82 | 0.67 | 0.61 |
| 20 | Nanjing, Jiangsu (Liaoning) | 0.85 | 0.69 | 0.68 | 0.60 |
| 21 | Shenzhen, Guangdong (unknown) | 0.76 | 0.66 | 0.69 | 0.61 |
| 22 | Nanjing, Jiangsu (Yunnan) | 0.73 | 0.60 | 0.68 | 0.64 |
| 23 | Shenzhen, Guangdong (unknown) | 0.30 | 0.26 | 0.95 | 0.87 |

Validation of the Quantitative Analysis: The method for quantitative analysis of four constituents in Fructus psoraleae was validated in terms of linearity, recovery and repeatability. Linearity was examined with standard solutions. The linear relationships between the injection quantities (μg, x-asis) and peak area ratio (y-axis) were expressed by the equations listed in Table 3. The correlation coefficients ranged from 0.9997 to 0.9999, and the calibration curves were straight lines. Quintuplicate samples were spiked with known amounts of these four compounds and then extracted in order to study the recoveries. The average recoveries ranged from 96.47 to 100.73%, and the ranges of relative standard deviations (RSD) were from 2.19 to 3.57%. Five individual samples from the same batch were extracted and processed in accordance with sample preparation procedures for quantitative analysis. The RSD of the determination results of these four constituents ranged from 1.98 to 3.03%. It indicated that the repeatability is suitable for quantitative analysis. The validation results (calibration equation, recovery and repeatability) are listed in Table 3.

TABLE 3
Validation data of the quantitative analysis of Fructus Psoraleae by HPLC

| | | Correlation | Average | Recovery | Repeatability |
|---|---|---|---|---|---|
| Compound | Calibration equation | coefficient | recovery (%) | RSD (%) | RSD (%) |
| 1 | y = 2949.3 x + 6.0649 | 0.9998 | 96.5 | 3.1 | 2.5 |
| 2 | y = 2924.7 x + 13.999 | 0.9999 | 97.1 | 3.6 | 3.0 |
| 3 | y = 6789.9 x − 14.593 | 0.9998 | 100.7 | 2.3 | 2.0 |
| 4 | y = 6714.4 x − 12.456 | 0.9997 | 99.3 | 2.2 | 2.0 |

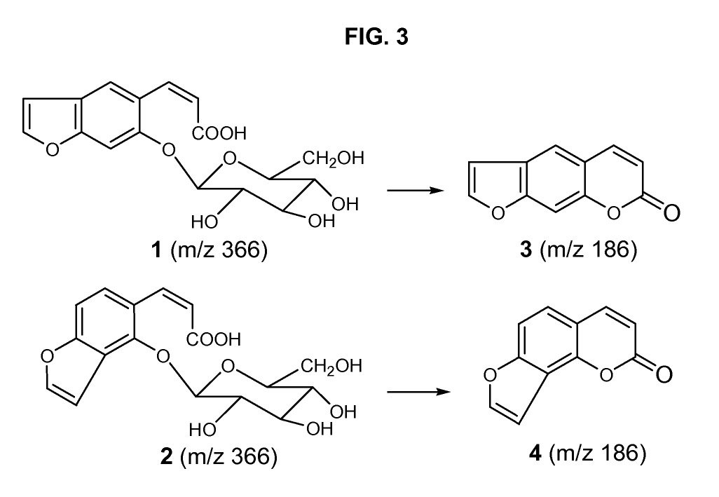

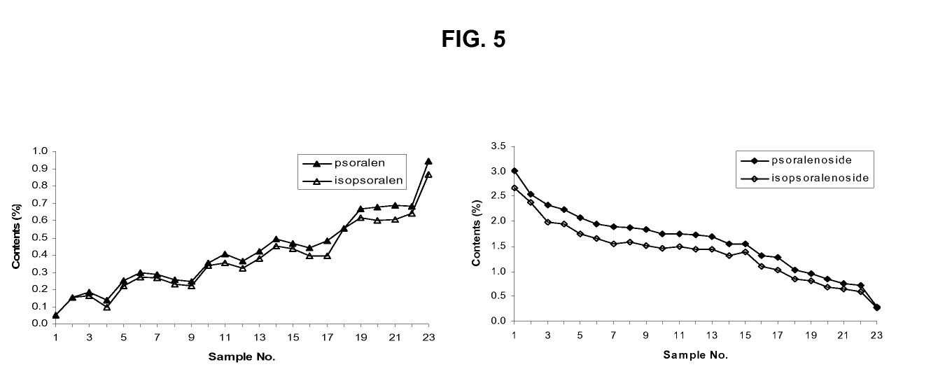

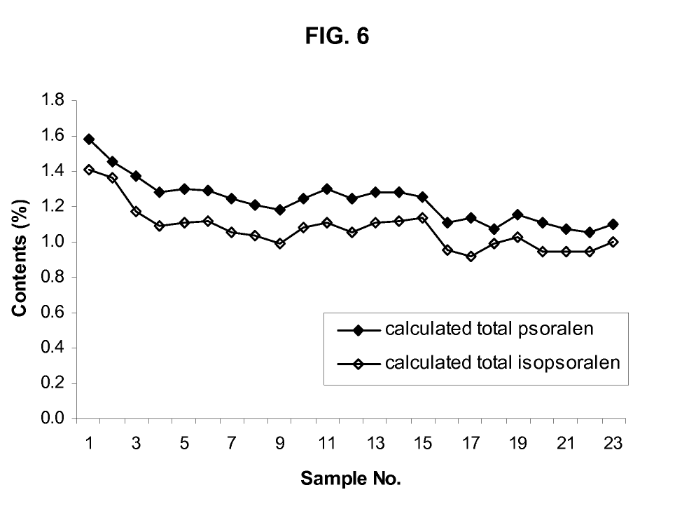

Contents of the Four Compounds in Tested Samples: FIG. 5 presents the contents of psoralenoside (1), isopsoralenoside (2), psoralen (3), and isopsoralen (4) in the 23 samples as determined by HPLC. The results show that the tested samples contained very different contents of psoralen (3), and isopsoralen (4), ranging from 0.05 to 0.95%, and from 0.05 to 0.87%, respectively. Sample No. 1 contained the lowest amount of coumarins 3 and 4, while sample No. 23 had the highest (almost twenty times higher). The coumarin contents were rising from sample No. 1 to No. 23. According to these results, sample No. 23 would usually be regarded as the best herbal material, and sample No. 1 would be the worst. However, the determination of the benzofuran glycoside led to a more interesting discovery. From sample No. 1 to No. 23, the contents of psoralenoside (1), and isopsoralenoside (2) decreased from 3.02 and 2.68% to 0.30 and 0.26%, respectively. A different conclusion regarding the herbal quality could be made on the basis of the contents of psoralenoside (1), and isopsoralenoside (2). As these different samples would be used interchangeably to exert similar therapeutic effects under the TMC, it would be more accurate to combine them as quality marks so that the quality assessment would be more consistent with clinical effects. Quality Assessment Standard of the Present Invention: A novel quality assessment standard (i.e., total content of the coumarins) was provided by present invention. This is supported by the biosynthetic relationship between coumarins 3 and 4 and the benzofuran glycosides 1 and 2 (FIG. 3). The content of the coumarins is a better quality standard that reflects more accurately the actual quality of the herbal material as used under TCM. The total content of coumarins has two components: determined content and potential contents. The potential content of coumarins were derived from the related benzofuran glycosides in a ratio of their molecular weight 186/366, e.g., CC_{1→3}=C₁×186/366. In this particular example, using the formulae Cₜₒₜₐₗ₋₃=C₃+CC_{1→3},the total content of compound 3 which includes the determined content (C₃) and the potential content was calculated as shown in Table 4. Similarly, the total content of compound 4 was also calculated. It was revealed by the calculated results that all twenty-three samples (FIG. 6) contained relatively consistent contents of coumarins 3 and 4 (about 1.20% and 1.05%, respectively). This suggested that the yielding time and storage conditions rather than the culturing area had considerable effects on the quality of Fructus Psoraleae, and that the quality would not be very changeable if a processing method was applied to convert all the glycosides into the coumarins. These two novel benzofuran glycosides, psoralenoside and isopsoralenoside, should be included as chemical markers in quality control and quality analysis for the herb Fructus Psoraleae.

TABLE 4
Converted contents (from 1 and 2 to 3 and 4) and total contents of
psoralen (3) and isopsoralen (4) in 23 batches of Fructus psoraleae

| | |
|---|---|
| Converted content | Total content |

| Sample | CC_{1→3} | CC_{2→4} | Cₜₒₜₐₗ₋₃ | Cₜₒₜₐₗ₋₄ |
|---|---|---|---|---|
| 1 | 1.53 | 1.36 | 1.58 | 1.41 |
| 2 | 1.30 | 1.21 | 1.45 | 1.37 |
| 3 | 1.19 | 1.01 | 1.37 | 1.17 |
| 4 | 1.14 | 1.00 | 1.28 | 1.09 |
| 5 | 1.05 | 0.89 | 1.30 | 1.11 |
| 6 | 0.99 | 0.84 | 1.29 | 1.12 |
| 7 | 0.96 | 0.79 | 1.25 | 1.06 |
| 8 | 0.95 | 0.81 | 1.21 | 1.04 |
| 9 | 0.94 | 0.77 | 1.18 | 0.99 |
| 10 | 0.89 | 0.74 | 1.24 | 1.08 |
| 11 | 0.89 | 0.76 | 1.30 | 1.11 |
| 12 | 0.88 | 0.74 | 1.25 | 1.06 |
| 13 | 0.86 | 0.73 | 1.28 | 1.11 |
| 14 | 0.79 | 0.67 | 1.28 | 1.12 |
| 15 | 0.79 | 0.70 | 1.25 | 1.14 |
| 16 | 0.67 | 0.56 | 1.11 | 0.95 |
| 17 | 0.65 | 0.52 | 1.14 | 0.92 |
| 18 | 0.52 | 0.43 | 1.07 | 0.99 |
| 19 | 0.48 | 0.42 | 1.15 | 1.03 |
| 20 | 0.43 | 0.35 | 1.11 | 0.95 |
| 21 | 0.38 | 0.33 | 1.07 | 0.94 |
| 22 | 0.37 | 0.30 | 1.05 | 0.95 |
| 23 | 0.15 | 0.13 | 1.10 | 1.00 |

Manufacturing Pharmaceutical Compositions Containing Psoralenoside and Isopsoralenoside:

As shown in the foregoing, psoralenoside and isopsoralenoside are major compounds in Fructus Psoraleae, a popular herbal material used under the TCM for thousands of years and it is contemplated, as people with ordinary skill in the art would do, that the two newly discovered compounds are ingredients contributing to the herbal's therapeutic effects and that it is desirable at times to prepare pharmaceutical composition from substantially pure psoralenoside and/or isopsoralenoside, which may isolated from herbals or through total or semi chemical synthesis. As it is the status of the art in the pharmaceutical industry, once substantially pure preparations of a compound are obtained, various pharmaceutical compositions or formulations can be prepared from the substantially pure compound using conventional processes or future developed processes in the industry. Specific processes of making pharmaceutical formulations and dosage forms (including, but not limited to, tablet, capsule, injection, syrup) from chemical compounds are not part of the invention and people of ordinary skill in the art of the pharmaceutical industry are capable of applying one or more processes established in the industry to the practice of the present invention. Alternatively, people of ordinary skill in the art may modify the existing conventional processes to better suit the compounds of the present invention. For example, the patent or patent application databases provided at USPTO official website contain rich resources concerning making pharmaceutical formulations and products from effective chemical compounds. Another useful source of information is Handbook of Pharmaceutical Manufacturing Formulations, edited by Sarfaraz K. Niazi and sold by Culinary & Hospitality Industry Publications Services.

It is further contemplated that the novel compounds psoralenoside and isopsoralenoside may be modified in various ways which are known in the art. Therefore the compounds of the present invention encompass all the compounds having the backbone structure defined by formula (1) or (2).

The term “pharmaceutical carrier” means an ingredient contained in a drug formulation that is not a medicinally active constituent. The term “an effective amount” refers to the amount that is sufficient to elicit a therapeutic effect on the treated subject. Effective doses will vary, as recognized by those skilled in the art, depending on the types of diseases treated, route of administration, excipient usage, and the possibility of co-usage with other therapeutic treatment. A person skilled in the art may determine an effective amount under a particular situation.

A “pharmaceutically acceptable carrier” is determined in part by the particular composition being administered and in part by the particular method used to administer the composition. A wide variety of conventional carrier may be suitable for pharmaceutical compositions of the present invention and can be selected by people with ordinary skill in the art.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

### REFERENCES

1. Chen, X, Kong, L., Su, X, Pan, C., Ye, M., Zou, H., J. Chromatogr. A., 1089, 87-100, (2005).
2. Zhao, L., Huang, C., Shan, Z., Xiang, B., Mei, L., J. Chromatogr. B., 821, 67-74, (2005).
3. Zhao, L. H., Wu, M. H., Xiang, B. R., Chem. Pharm. Bull. 53, 1054- 1057, (2005).
4. Liu, R., Li, A., Sun, A., Kong, L., J. Chromatogr. A., 1057,225-228, (2005).
5. Yin, S., Fan, C. Q., Wang, Y, Dong, L., Yue, J. M., Bioorg. & Med.Chem., 12, 387-4392, (2005).
6. Chen, Y, Kong, L. D., Xia, X, Kung, H. F., Zhang, L., J. Ethnopharm., 96, 451-459, (2005).
7. Guo, J., Weng, X, Wu, H., Li, Q., Bi, K, Food Chem., 91, 287-292, (2005).
8. Zhang, C. Z., Wang, S. X, Zhang, Y, Chen, J. P., Liang, X M., J. Ethnopharm., 96, 295-300, (2005).
9. Tang, S. Y., Whiteman, M., Peng, Z. F., Jenner, A., Yong, E. L., Halliwell, B., Free Rad. Biol. & Med., 36, 1575-1587, (2004).
10. Prasad, N. R., Anandi, C., Balasubramanian, S., Pugalendi, K. V., J. Ethnopharm., 91, 21-24, (2004).
11. Khatune, N. A. M., Islam, E., Haque, M. E., Khondkar, Rahman, P., M. M., Fitoterapia, 75, 228-230, (2004).
12. Takizawa, T., Mitsumori, K, Takagi, H., Nasu, M., Yasuhara, K, Onodera, H., Imai, T., Hirose, M., Food & Chem. Toxicol., 42,1-7, (2004).
13. Whelan, L. C., Ryan, M. F., Phytomedicine, 10, 53-58, (2003).
14. Qamaruddin, A., Parveen, N., Khan, N. U., Singhal, K. C., J. Ethnopharm., 82, 23-28, (2002).
15. Sandra M. Newton, Clara Lau, Sudagar S. Gurcha, Gurdyal S. Besra and Colin W. Wright J. Ethnopharm., 79, 57-67, (2002).
16. Pae, H. O., Cho, H., Oh, G. S., Kim, N. Y., Song, E. K., Kim, Y. C., Yun, Y. G., Kang, C. L., Kim, J. D., Kim, J. M., Chung, H. T., Intern. Immunopharm., 1, 1849-1855, (2001).
17. Sampson, J. H., Raman, A., Karlsen, G., Navsaria, H., Leigh, I. M., Phytomedicine, 8, 230-235 (2001).
18. Panikkar, K. R., Latha, P. G., J. Ethnopharm., 68, 295-298, (1999).
19. Hamed, A. I., Sparinguel, I. V., El-Emary, N. A., Phytochemistry, 50, 887-890, (1999).

What is claimed is:

1. A compound, comprising a backbone structure defined by formula (1) or (2):

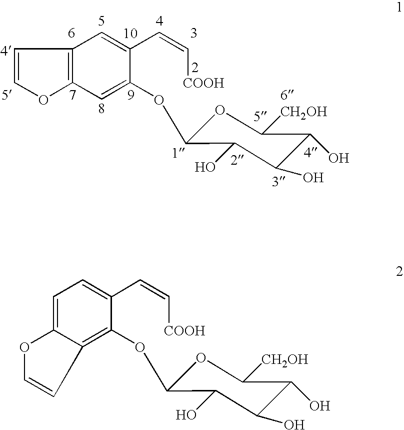

2. The compound of claim 1, wherein said backbone structure is defined by formula (1).

3. The compound of claim 1, wherein said backbone structure is defined by formula (2).

4. The compound of claim 2, which is psoralenoside.

5. The compound of claim 3, which is isopsoralenoside.

6. A pharmaceutical composition, which comprises a pharmaceutically acceptable carrier and an effective amount of a compound of claim 1.

7. The pharmaceutical composition of claim 6, wherein said compound comprises a backbone structure of formula (1).

8. The pharmaceutical composition of claim 6, wherein said compound comprises a backbone structure of formula (2).

9. The pharmaceutical composition of claim 6, wherein said compound is psoralenoside.

10. The pharmaceutical composition of claim 6, wherein said compound is isopsoralenoside.

11. A method for assessing quality of an herbal medicine of Fructus Psoraleae, comprising a step of measuring the content of a compound selected from the group consisting of psoralenoside and isopsoralenoside.

12. The method of claim 11, wherein said compound is psoralenoside.

13. The method of claim 11, wherein said compound is isopsoralenoside.

14. A method of isolating a chemical ingredient from an herbal medicine of Fructus Psoraleae, comprising a step of (a) moistening said herbal medicine of Fructus Psoraleae with a first solvent which is substantially free of water, (b) drying said moistened herbal medicine and (c) then extracting said herbal medicine with a second solvent which is water or an alcohol.

15. The method of claim 14, wherein said first solvent is selected from the group consisting of hexane, cyclohexane, Petroleum ether, benzene, chloroform, Diethyl ether, ethyl acetate, n-butanol, acetone, ethanol, and acetonitrile.

16. The method of claim 15, wherein said chemical ingredient is psoralenoside.

17. The method of claim 15, wherein said chemical ingredient is isopsoralenoside.

18. The method of claim 14, wherein said second solvent is water.

19. The method of claim 14, wherein said second solvent is ethanol.

20. The method of claim 14, wherein said first solvent is which is EtOAc and said second solvent is MeOH.
